SEQUENCE LISTING (4 sequences)

SEQ ID NO: 1 - DNA, 15 bp, Homo sapiens
ctgcccggcgagtcg

SEQ ID NO: 2 - DNA, 22 bp, Homo sapiens
ccgtcttcctccatctcatagc

SEQ ID NO: 3 - DNA, 29 bp, Homo sapiens
aaggtaattatgtggtgacagatcacggc

SEQ ID NO: 4 - protein, 14 aa, Homo sapiens
LEEKKGNYVVTDHS

(19) United States
(12) Patent Application Publication
(10) Pub. No.: US 2015/0307614 A1
(43) Pub. Date: Oct. 29, 2015

(54) Enhanced immunological responses

(71) Applicant: Duke University, Durham, NC (US)

(72) Inventors: John H. Sampson, Durham, NC (US); Duane A. Mitchell, Gainesville, FL (US); Peter E. Fecci, Boston, MA (US)

(21) Appl. No.: 14/545,016

(22) Filed: Mar. 17, 2015

Related U.S. Application Data
(63) Continuation of application No. 13/336,590, filed on Dec. 23, 2011, now abandoned.
Application No. 13/336,590 is a continuation-in-part of application No. 12/947,362, filed on Nov. 16, 2010.
(60) Provisional application No. 61/261,476, filed on Nov. 16, 2009.

(51) Int. Cl. C07K 16/28; A61K 45/06; A61K 39/395

(52) U.S. Cl. CPC C07K 16/2866; A61K 39/3955; A61K 45/06; C07K 2317/76

(57) ABSTRACT

Cancers and other diseases can be treated with two or three types of agents: an agent which induces lymphodepletion or lymphopenia; an inhibitory antibody to a surface marker on Treg cells; and optionally a specific antigen. This combination may lead to enhanced immune responses despite lymphodepletion or lymphopenia.

This application is a continuation of U.S. application Ser. No. 13/336,590, filed Dec. 23, 2011, which claims the benefit of U.S. application Ser. No. 12/947,362 filed Nov. 16, 2010, and 61/261,476 filed Nov. 16, 2009, the contents of which are expressly incorporated by reference.

The U.S. government provided funds used in making the present invention. It retains rights in the invention as provided under the provisions of grant no. 2R21CA132891-05.

### TECHNICAL FIELD OF THE INVENTION

This invention is related to the area of immunotherapy. In particular, it relates to enhancing antigen specific immune responses.

### BACKGROUND OF THE INVENTION

Malignant brain tumors are the most common cause of death among children, and account for more deaths in adults than melanoma. Conventional therapy is severely constrained by the need to eradicate tumor cells that are hidden behind a restrictive blood-brain barrier or that have invaded eloquent brain tissue. As a result, surgery and radiation must be curtailed to avoid incapacitating collateral damage, and chemotherapy becomes toxic to rapidly dividing extracerebral normal tissues before eliminating all intracerebral tumor cells. The immune system, however, has the potential capacity to eliminate the altered neoplastic cells with incredible specificity.

A consistent in-frame deletion in the extra-cellular domain of the epidermal growth factor receptor (EGFRvIII) produces a constitutively active tyrosine kinase that enhances neoplastic cell growth and migration and confers radiation and chemotherapeutic resistance to tumor cells in patients with glioblastoma multiforme (GBM) and a broad array of other common cancers. It also results in a truly tumor-specific target amenable to immunotherapeutic attack.

Despite aggressive surgical resection, high-dose focused radiation therapy, and chemotherapy, patients diagnosed with GBM have a median survival of less than 15 months after diagnosis (Stupp et al., Optimal role of temozolomide in the treatment of malignant gliomas. Curr Neurol Neurosci Rep. 2005 May; 5(3):198-206.). Failure of therapy can be attributed, at least in part, to a relatively narrow therapeutic index so that attempts at dose escalation results in dose-limiting systemic or neurological toxicity. The use of immunotherapy has held promise for the potential treatment of these tumors but until recently, few have demonstrated clinical efficacy. Several clinical trials, with selected patients, involving vaccination of glioma patients with dendritic cells (DCs) and either acid-eluted peptides (Ashkenazi et al., A selective impairment of the IL-2 system in lymphocytes of patients with glioblastomas: increased level of soluble IL-2R and reduced protein tyrosine phosphorylation. Neuroimmunomodulation. 1997; Kolenko et al., Tumor-induced suppression of T lymphocyte proliferation coincides with inhibition of Jak3 expression and IL-2 receptor signaling: role of soluble products from human renal cell carcinomas. J Immunol. 1997 Sep. 15; 159(6):3057-67; Liau et al., Dendritic cell vaccination in glioblastoma patients induces systemic and intracranial T-cell responses modulated by the local central nervous system tumor microenvironment. Clin Cancer Res. 2005 Aug. 1; 11(15):5515-25) or an antigen-specific peptide (Heimberger A B, Archer G E, et al., Dendritic cells pulsed with a tumor-specific peptide induce long-lasting immunity and are effective against murine intracerebral melanoma. Neurosurgery. 2002 January; 50(1):158-64; discussion 164-6) have demonstrated promise by increasing median survival time to a range of 20-31 months. Furthermore, in a recently completed phase II clinical trial utilizing an antigen-specific immunotherapeutic approach, time to progression (TTP) in GBM patients was delayed to 15 months, which is in marked contrast to the standard of care consisting of radiotherapy and temozolomide that had a TTP of 7 months (Stupp et al., 2005, supra), and median survival was 29 months (Heimberger et al, J Transl Med. 2005 Oct. 19; 3:38 The natural history of EGFR and EGFRvIII in glioblastoma patients.). Cumulatively, these immunotherapy trials suggest that despite the inherent immunosuppression of malignant glioma patients, efficacious immune responses can be generated. However, there is reluctance to not treat GBM patients with some form of chemotherapy given the recently established standard of care and the overall poor prognosis.

There is a continuing need in the art to develop better methods for using immunotherapy to treat diseases in general, including tumors such as brain tumors including glioblastomas.

### SUMMARY OF THE INVENTION

Applicants provide a method of treating a lymphodepleted or lymphopenic subject. Sufficient inhibitory antibody to IL-2Rα to inhibit Treg cells is administered to the lymphodepleted or lymphopenic subject. An immune response to a specific antigen is thereby increased.

Similarly, a kit is provided for use in subjects lymphodepleted or lymphopenic by disease or irradiation. The kit comprises an inhibitory antibody to IL-2Rα and an immunogen.

Applicants provide a method of treating a tumor in a human subject. An amount of an inhibitory antibody to IL-2Rα sufficient to inhibit Treg cells is administered to the human subject. An immune response to the tumor is thereby increased. The immune response may be endogenous or vaccine-induced. The subject may be lymphopenic due to receipt of chemotherapy or due to the biological effects of the tumor. The subject may be a recipient of an anti-tumor vaccine, before, during, or after treatment with the inhibitory antibody. Exemplary anti-tumor vaccines are peptide vaccines, such as EGFRvIII vaccines.

Applicants provide a method for treating a tumor in a subject. An effective amount of each of a chemotherapeutic agent which induces lymphopenia; an inhibitory antibody to a surface marker on Treg cells; and an anti-cancer vaccine are administered to the subject. The lymphopenia-inducing chemotherapeutic agent may optionally be temozolamide. The inhibitory antibody may optionally be daclizumab. The anti-cancer vaccine may optionally be an EGFRvIII peptide vaccine.

According to another embodiment a method is provided for treating a tumor in a subject. An effective amount of each of an EGFRvIII peptide, conjugated to KLH; daclizumab; and an alkylating agent are administered to the subject.

According to still another embodiment, a method is provided for treating a tumor in a subject. An effective amount of each of an EGFRvIII peptide conjugated to KLH; daclizumab; and a chemotherapeutic agent which induces lymphopenia are administered to the subject.

According to still another embodiment, a method is provided for treating a tumor in a subject. An effective amount of each of temozolomide; an inhibitory antibody to a surface marker on Treg cells; and an EGFRvIII peptide conjugated to KLH are administered to the subject.

Another aspect of the invention is a method of treating a tumor in a human subject. An effective amount of each of temozolamide; daclizumab; and an anti-cancer vaccine are administered to the subject.

Another aspect of the invention is a kit for use in treating tumors. The kit comprises in a container: temozolamide; daclizumab; and an anticancer vaccine.

These and other embodiments that will be apparent to those of skill in the art upon reading the specification provide the art with additional methods and tools for treating diseases, including treatment-refractory tumors.

### BRIEF DESCRIPTION OF THE DRAWINGS

FIG. 1 shows selective blockade of regulatory T cells using anti-CD25 monoclonal antibodies during lymphopenia. The mice were lymphodepleted using total body irradiation.

### DETAILED DESCRIPTION OF THE INVENTION

Applicants have found that lymphodepleted or lymphopenic subjects, but not untreated subjects, maintain antigen-specific T cell responses upon antibody blockade of CD25. The antigen-specific T cell responses can be induced, for example, by adoptive lymphocyte transfer or vaccination. Lymphodepletion can be accomplished by any means known in the art, including total body irradiation, chemotherapy, or as a result of a disease process, such as leukemia or HIV/AIDS. Alternatively, lymphodepletion can be accomplished by administering an antibody which specifically binds to lymphocytes.

CD25/IL-2Rα blockade can be used to enhance a cellular immune response or a humoral immune response. The response can be generated to an antigen that is administered as part of a vaccine or can be generated to an antigen that is present in the body, such as on an infectious agent or a pathological endogenous tissue. The enhanced immune response can overcome immune tolerance, in some instances. Vaccines for inducing humoral and/or cellular immune responses are known in the art. The antigen may be an antigen expressed on a tumor, e.g., glioblastoma multiforme. The antigen may be a non-tumor antigen. It may be desirable, in certain circumstances that the specific antigen (vaccine) and the inhibitory antibody be administered concomitantly. Concomitant administration may be via an admixture of the two substances. Concomitant administration may be via separate administrations within 1 hour, 1 day, or 1 week, for example.

CD25/IL-2Rα blockade can be achieved using any antibody that is specific for CD25/IL-2Rα. The antibody may be unarmed, i.e., lacking in any added cytotoxic agent. Exemplary of such antibodies are daclizumab and basiliximab, (a genetically engineered IgG-1, and a chimeric engineered IgG-1, respectively). The CD25/IL-2Rα inhibitory antibody can be administered at nadir or early recovery from nadir of lymphodepletion.

Repeated cycles of lymphodepletion, CD25/IL-2Rα blockade, and optionally immunization may be used in a patient to achieve increased immune response or to achieve a longer lasting immune response. Monitoring of the immune response can be accomplished by any means known in the art.

In order to simplify the use of the method in subjects lymphodepleted by disease or irradiation, kits can be assembled of components and/or reagents for accomplishing the method. Such a kit typically will comprise, in a single or divided container, an inhibitory antibody to IL-2Rα and an immunogen for inducing an immune response. The kit may or may not further contain a chemotherapeutic agent, which may have the effect of inducing lymphopenia. The kit may or may not contain temozolomide as such a chemotherapeutic agent.

Applicants have also found that administration of three agents to tumor-bearing human subjects may lead to synergistic effects. The concurrent administration of chemotherapy and immunotherapy has been considered a contraindication because of the concern that the chemotherapy-induced lymphopenia would ablate therapeutic efficacy of immunotherapy. Temozolomide has been shown to be an effective chemotherapeutic for patients with malignant gliomas and to deprive patients with glioblastoma (GBM) patients of this agent in order to treat with immunotherapy is controversial. Despite conventional dogma, the inventors demonstrate that both chemotherapy and immunotherapy can be delivered concurrently without negating the effects of immunotherapy. Surprisingly, temozolomide induced lymphopenia may actually be synergistic with a peptide vaccine. Moroever, use of an inhibitory antibody to a surface antigen on Tregs may further enhance the immunotherapeutic effect. The effect may involve effector cytotoxic CD8⁺ T cells and/or humoral antibodies. Other mechanisms may also be involved.

Daclizumab is an antibody typically used to induce immunosuppression by eliminating or inhibiting activated T-cells via antibody-induced killing. Applicants demonstrate in our animal models that such IL-2 receptor alpha-blocking antibodies can rather act by blocking signaling, and in the context of chemotherapy-induced lymphopenia, can actually have a counter-intuitive effect on immunosuppressive regulatory T-cells leading to immune enhancement. We have also shown this counter-intuitive effect in humans. Thus, antibodies that block IL-2 receptor alpha, such as daclizumab, can be used as immunostimulatory molecules. This is useful in the context of patients with cancer because they almost universally have increased regulatory T-cells or T_{Regs} that inhibit endogenous and anti-cancer vaccine-induced immune responses. Eliminating regulatory T-cells significantly enhances vaccination immune responses and anti-tumor efficacy. Antibodies which may be used include murine, humanized, human, chimeric, polyclonal and monoclonal.

“EGFRvIII” or “Epidermal Growth Factor Receptor mutation III” is a known mutant form of the Epidermal Growth Factor Receptor. See, e.g., U.S. Pat. No. 6,503,503.; see also U.S. Pat. Nos. 6,900,221; 6,673,602; 6,479,286; and 6,129,915, the contents of which are expressly incorporated herein. The mutation which causes the production of the vIII protein is typically characterized by a consistent and tumor-specific in-frame deletion of 801 base pairs from the extracellular domain that splits a codon and produces a novel glycine at the fusion junction.

“EGFRvIII peptide” as used herein refers to a peptide of suitable length, e.g., at least 10 or 12 amino acids, and up to 16, 20 or 30 amino acids, or more, which spans the mutated splice junction of the corresponding EGFRvIII protein. Examples include but are not limited to: H-LEEKKGNYVVTDHS-OH (SEQ ID NO:4), or “PEP-3.” The EGFRvIII peptide may be from (or correspond in sequence to) the EGFRvIII of any mammalian species, but is preferably human. Particular wild-type sequences of EGFR are shown in SEQ ID NO: 6 to 9 of WO 2007/056061.

Other proteins and polypeptides which may be used as cancer anti-vaccines include tumor-specific and tumor-associated proteins. Whole cell cancer vaccines may also be used. CMV (cytomegalovirus) proteins are useful for treating cancers such as glioblastoma multiforme (GBM), as these serve as a refuge for CMV. One particular CMV protein which may be used as a vaccine is pp65. Others can be used as well. Epstein Barr Virus (EBV) and human papilloma virus (HPV) proteins and polypeptides can also be used for treating tumors. Proteins that are mutated in tumors, overexpressed in tumors, associated with tumors may be used as tumor peptide vaccines as well. Examples of such antigens include adipophilin; AIM-2; ALDH1A1; BCLX (L); BING-4; CALCA; CPSF; cyclin D1; DKK1; ENAH (hMena); Ep-CAM; EphA3; EZH2; FGF5; G250/MN/CAIX; HER-2/neu; IL13Ralpha2; Intestinal carboxyl esterase; alpha-foetoprotein; M-CSF; MCSP; mdm-2; Meloe; MMP-2; MMP-7; MUC1; p53; PAX5; PBF; PRAME; PSMA; RAGE-1; RGS5; RhoC; RNF43; RU2AS; secernin 1; SOX10; STEAP1; survivin; Telomerase; VEGF; WT1; CEA; gp100/Pme117; Kallikrein 4; mammaglobin-A; Melan-A/MART-1; NY-BR-1; OA1; PSA; RAB38/NY-MEL-1; TRP-1/gp75; TRP-2; tyrosinase; BAGE-1; GAGE-1,2,8; GAGE-3,4,5,6,7; GnTVf; HERV-K-MEL; KK-LC-1; KM-HN-1; LAGE-1; MAGE-A1; MAGE-A2; MAGE-A3; MAGE-A4; MAGE-A6; MAGE-A9; MAGE-A10; MAGE-A12; MAGE-C2; mucin k; NA88-A; NY-ESO-1/LAGE-2; SAGE; Sp17; SSX-2; SSX-4; TAG-1; TAG-2; TRAG-3; TRP2-INT2g; XAGE-1b; alpha-actinin-4; ARTC1; BCR-ABL fusion protein (b3a2); B-RAF; CASP-5; CASP-8; beta-catenin; Cdc27; CDK4; CDKN2A; COA-1; dek-can fusion protein; EFTUD2; Elongation factor 2; ETV6-AML1 fusion protein; FLT3-ITD; FN1; GPNMB; LDLR-fucosyltransferaseAS fusion protein; HLA-A2d; HLA-A11d; hsp70-2; KIAAO205; MART2; ME1; MUM-1f; MUM-2; MUM-3; neo-PAP; Myosin class I; NFYC; OGT; OS-9; p53; pml-RARalpha fusion protein; PRDX5; PTPRK; K-ras; N-ras; RBAF600; SIRT2; SNRPD1; SYT-SSX1 or -SSX2 fusion protein; TGF-betaRII; and Triosephosphate Isomerase. In one embodiment, the tumor antigens are delivered by/as RNA-transfected Dendritic Cells. The RNAs may include a LAMP sequence to facilitate proper antigen processing.

“Carrier protein” refers to a protein which does not possess high homology to a protein found in the species that is receiving a composition of the invention and elicits an immune response. A protein possesses high homology if it is at least 75% identical, more preferably at least 85% identical or at least 90% identical to a protein as determined by any known mathematical algorithm utilized for the comparison of two amino acid sequences (see, e.g., Karlin and Altschul, 1990, Proc. Natl. Acad. Sci. USA 87: 2264-2268; Karlin and Altschul, 1993, Proc. Natl. Acad. Sci. USA 90: 5873-5877; Torellis and Robotti, 1994, Comput. Appl. Biosci. 10: 3-5; and Pearson and Lipman, 1988, Proc. Natl. Acad. Sci. 85: 2444-8). Preferably, the percent identity of two amino acid sequences is determined by BLAST protein searches with the XBLAST program, score=50, word length=3. Examples of heterologous carrier proteins include, but are not limited to, KLH, PhoE, mLT, TraT, or gD from BhV-1 virus. See, e.g., U.S. Pat. No. 6,887,472. Such carrier proteins may be conjugated or linked to the tumor antigen directly or by an intervening linker segment such as a chain of one or more (e.g., 2, 4, 6) intervening amino acids (e.g., an intervening CYS residue) in accordance with known techniques.

“KLH” or “keyhole-limpet hemocyanin” is a known carrier protein to which another protein may be conjugated in accordance with known techniques. See, e.g., U.S. Pat. No. 6,911,204.

“Adjuvant” refers to any one of a diverse class of compounds that enhance the therapeutic efficacy of a vaccine which is administered concurrently with the adjuvant. In some embodiments the adjuvant is a hematopoietic growth factor such as GM-CSF. Common examples of adjuvants include but are not limited to aluminium hydroxide, -phosphate or -oxide, oil-in-water or water-in-oil emulsion based on, for example a mineral oil, such as Bayol Fo or Marcol 52™ or a vegetable oil such as vitamin E acetate, saponins, BCG, M vaccae, Tetanus toxoid, Diphtheria toxoid, Bordetella pertussis, interleukin 2, interleukin 12, interleukin 4, interleukin 7, Complete Freund's Adjuvant, Incomplete Freund's Adjuvant, and a nonspecific adjuvant. See, e.g., U.S. Pat. No. 6,699,483.

“Hematopoietic growth factors” or “HGFs” are known. See, e.g., U.S. Pat. No. 6,863,885. In general, HGFs are glycoprotein cytokines that regulate the proliferation and differentiation of hematopoietic progenitor cells. The hematopoietic growth factors intended to be used in the present invention can be selected from the group G-CSF (granulocyte colony stimulating factor), SCF (stem cell factor), GM-CSF (granulocyte macrophage colony stimulating factor), IL-1 (interleukin-1), IL-3, IL-6, IL-8, IL-11, IL-12, LIF (leukemia inhibitory factor), FGF-beta (fibroblast growth factor beta), FLT3, or a combination thereof. These growth factors can be purchased (e.g., R&D Systems, Minneapolis, Minn.) or made following procedures set forth in the art generally and in publications describing the factors. Additionally, the hematopoietic growth factor can be a modified form of the factor or a fusion protein of hematopoietic growth factors selected from the group GCSF, SCF, GM-CSF, IL-I, IL-3, IL-6, IL-8, IL-11, IL-12, LIF, FGF-beta, and FLT3. HGFs include modified growth factors (e.g., muteins) and fusion proteins, which can be made according to methods known in the art. See, e.g. (Sambrook et al., Molecular Cloning: A Laboratory Manual, 2nd Ed., Cold Spring Harbor Laboratory, Cold Spring Harbor, N.Y., 1989). Hematopoietic growth factors that stimulate macrophage function such as GM-CSF are particularly preferred. These can be used as adjuvants.

“External beam radiotherapy” can be carried out by delivering a beam of high-energy x-rays to the location of the patient's tumor. The beam is generated outside the patient and is targeted at the tumor site. No radioactive sources are placed inside the patient's body. This can be used in conjunction with any other treatment step according to the invention.

“Total body irradiation” is used typically as a preparation for a stem cell or bone marrow transplantation. It is often used to destroy the immune system of the host prior to reconstitution with the transplanted stem cells. The total body irradiation may suppress transplant rejection and may also kill residual cancer cells in the subject.

“Treat” refers to any type of treatment or prevention that imparts a benefit to a subject afflicted with a disease or at risk of developing the disease, including improvement in the condition of the subject (e.g., in one or more symptoms), delay in the progression of the disease, delay the onset of symptoms or slow the progression of symptoms, etc. The term “treatment” also includes prophylactic treatment of the subject to prevent the onset of symptoms.

“Treatment” and “prevention” are not meant to imply cure or complete abatement of symptoms. Rather, these refer to any type of treatment that imparts a benefit to a patient afflicted with a disease, including improvement in the condition of the patient (e.g., in one or more symptoms), delay in the progression of the disease, etc.

“Treatment-effective amount” means an amount of the antibody sufficient to produce a desirable effect upon a patient inflicted with cancer such as glioblastoma, including improvement in the condition of the patient (e.g., in one or more symptoms), delay in the progression of the disease, etc.

“Kit” describes a collection of separate or combined elements that are within a single container, such as a box, envelope, bottle, jar, etc. The elements of the kit may be in separate subcontainers or they may be admixed. Instructions for using, storing, reconstituting, etc. may also be included, either within the container, upon the container, or at a separate referenced location, such as at a website or other archive or library of information. The kit may be for single use, or for multiple administrations, or for multiple patients. The kit may further comprise diagnostic reagents to test and/or confirm that the subject will be an appropriate subject for the treatment.

Subjects in need of treatment by the methods described here may include subjects afflicted with glioblastoma or astrocytoma, as well as subjects afflicted with other solid tumors or cancers such as lung, colon, breast, brain, liver, prostate, spleen, muscle, ovary, pancreas, head and neck, skin (including melanoma), etc. Subjects in need of treatment particularly include subjects afflicted with a tumor, such as a brain tumor, that expresses EGFRvIII. The tumor may be a primary tumor, a metastatic tumor, or a recurrent tumor. Subjects to be treated by the methods of the invention particularly include subjects afflicted with a tumor expressing EGFRvIII, including gliomas, fibrosarcomas, osteosarcomas, melanoma, Wilms tumor, colon carcinoma, mammary and lung carcinomas, and squamous carcinomas. Subjects to be treated by the present invention most particularly include subjects afflicted with brain tumors or cancers, such as glioblastomas, particularly glioblastoma multiforme, and cystic astrocytoma.

Lymphopenia is a condition in which there is a lower-than-normal number of lymphocytes (a type of white blood cell) in the blood. It is also known as lymphocytic leukopenia and lymphocytopenia. Chemotherapeutic agents which induce lymphopenia include capecitabine, temozolomide, topiramate, requip, bortezomib, thalidomide, risperidone, paroxetine (paroxetine hydrochloride), pamidronate disodium, irbesartan, citalopram (citalopram hydrobromide), ceftriaxone (ceftriaxone sodium), rifapentine, ultiva, exemestane, relenza, olsalazine, cefpodoxime, sutent, imatinib, mesylate, riluzole, ribavirin, micafungin, sodium oxaliplatin, levofloxacin, mustargen, irinotecan hydrochloride, docetaxel, chlorambucil, Cefaclor, nexavar, alendronic acid (alendronate sodium), ziagen doxorubicin hydrochloride, cladribine, and valdecoxib. Any such agents can be used, as indicated for the particular tumor and patient characteristics. Alkylating agents which can be used include: nitrogen mustards, such as mechlorethamine (nitrogen mustard), chlorambucil, cyclophosphamide (Cytoxan®), ifosfamide, and melphalan, nitrosoureas, which include streptozocin, carmustine (BCNU), and lomustine, alkyl sulfonates, such as busulfan, triazines, such as dacarbazine (DTIC), and temozolomide (Temodar®), ethylenimines, such as thiotepa and altretamine (hexamethylmelamine). Lymphopenia and lymphodepletion are used interchangeably to describe the state of reduced lymphocyte number.

Antibodies which block binding to surface receptors on Treg cells include those that bind to IL-2R alpha (CD25), as well as antibodies that bind to neuropilin-1, CD39, CTLA-4, GITR, LAG3, GPR83, Folate receptor 4, PD1, and ICOS. One particular antibody which can be used is daclizumab, which is commercially available. Other antibodies to IL-2R alpha can be used, as well as antibodies to other Treg cell surface receptors.

The present invention is primarily concerned with the treatment of human subjects, including male and female subjects and neonatal, infant, juvenile, adolescent, adult, and geriatric subjects, but the invention may also be carried out on animal subjects, particularly mammalian subjects such as mice, rats, dogs, cats, livestock and horses for veterinary purposes, and for drug screening and drug development purposes.

The pharmaceutical compositions of the invention can be prepared in accordance with known techniques. Typically, the active agents are included in a pharmaceutically acceptable carrier. A variety of aqueous carriers may be used, e.g., water, buffered water, 0.9% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as buffering agents, tonicity adjusting agents, wetting agents and the like, for example, sodium acetate, sodium lactate, sodium chloride, potassium chloride, calcium chloride, sorbitan monolaurate, triethanolamine oleate, etc.

The compositions and methods of the invention may include the administration of one or more co-adjuvants. Suitable co-adjuvants include, but are not limited to: (1) aluminum salts (alum), such as aluminum hydroxide, aluminum phosphate, aluminum sulfate, etc.; (2) oil-inwater emulsion formulations (with or without other specific immunostimulating agents such as muramyl peptides (see below) or bacterial cell wall components), such as for example (a) MF59 (PCT Publication No. WO 90/14837), containing 5% Squalene, 0.5% Tween 80, and 0.5% Span 85 formulated into submicron particles, (b) SAF, containing 10% Squalane, 0.4% Tween 80, 5% pluronic-blocked polymer L121, and thr-MDP (see below) either microfluidized into a submicron emulsion or vortexed to generate a larger particle size emulsion, and (c) Ribi^{TM} adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™) (for a further discussion of suitable submicron oil-in-water emulsions for use herein, see PCT Publication No. WO 99/30739, published Jun. 24, 1999); (3) saponin adjuvants, such as Stimulon™ (Cambridge Bioscience, Worcester, Mass.) may be used or particle generated therefrom such as ISCOMs (immunostimulating complexes); (4) Complete Freunds Adjuvant (CF A) and Incomplete Freunds Adjuvant (IF A); (5) cytokines, such as interleukins (IL-1, IL-2, etc.), macrophage colony stimulating factor (M-CSF), tumor necrosis factor (TNF), etc.; (6) detoxified mutants of a bacterial ADP-ribosylating toxin such as a cholera toxin (CT), a pertussis toxin (PT), or an E. coli heat-labile toxin (LT), particularly LT-K63 (where lysine is substituted for the wild-type amino acid at position 63) LT-R72 (where arginine is substituted for the wild-type amino acid at position 72), CT-SI09 (where serine is substituted for the wild-type amino acid at position 109), adjuvants derived from the CpG family of molecules, CpG dinucleotides and synthetic oligonucleotides which comprise CpG motifs (see, e.g., Krieg et al., Nature, 374:546 (1995) and Davis et al., J. Immunol., 160:870-876 (1998)) and PT-K9/GI29 (where lysine is substituted for the wild-type amino acid at position 9 and glycine substituted at position 129) (see, e.g., PCT Publication Nos. W093/13202 and W092/19265); (7) other substances that act as immunostimulating agents to enhance the effectiveness of the composition. See, e.g., U.S. Pat. No. 6,534,064; and (8) other ligands for Toll-like receptors in addition to CpG and RIBI adjuvants, such as bacterial flagellin (an effective adjuvant for CD4+ T cells; see McSorley et al., J. immunol. 169: 3914-9 (October 2002).

The active agents may be administered by any medically appropriate procedure, e.g., normal intravenous or intra-arterial administration, injection into the cerebrospinal fluid). In certain cases, intradermal, intracavity, intrathecal or direct administration to the tumor or to an artery supplying the tumor is advantageous. Where the tumor or a portion thereof has been previously surgically removed the treatment agents may be administered into the site of the tumor (and particularly into an enclosed cavity or “resection cavity” at the site of the tumor) by direct injection or through a pre-implanted reservoir.

Dosage of the active agents will depend on, among other things, the condition of the subject, the particular category or type of cancer being treated, the route of administration, the nature of the therapeutic agent employed, and the sensitivity of the tumor to the particular therapeutic agent.

In general, the dose of the tumor antigen or vaccine, such as EGFRvIII, including any carrier protein or peptide conjugated thereto, will be from 10, 100 or 500 μg up to 2 or 3 mg per subject, for each dose. Doses may be given on a single occasion, optionally including follow-up or “booster” doses (e.g., one, two or three follow up or “booster” dosages given at intervals of from one to three weeks). Note that doses can be divided, such as administering to different injection sites, to reduce side effects such as local responses, if desired. Where the formulation contains both tumor antigen bound (or “conjugated”) to the carrier protein and tumor antigen free of the carrier protein, the calculated dosage can include both the amount of both bound and free tumor antigen and carrier protein.

In general, the dose of the adjuvant such as GM-CSF will also be from 10 or 20 μg up to 500 μg, or 1 or 2 mg per subject, administered on the same schedule or different schedule from the dose of the tumor antigen. When administered on the same schedule the adjuvant may be administered in the same carrier as the tumor antigen. When not combined in the same carrier, the dose of adjuvant need only be administered sufficiently close in time to the dose of tumor antigen to enhance the efficacy thereof (e.g., within one or two hours; on the same day; etc.).

Alkylating agents, a subset of chemotherapeutic agents useful for carrying out the present invention, include (but are not limited to) 1,3-bis(2-chloroethyl)-1-nitrosourea (BCNU) and tetrazine derivatives, particularly [3H]imidazo[5,1-d]1,2,3,5-tetrazin-4-one derivatives such as temozolomide and analogs thereof (including pharmaceutically acceptable salts and pro drugs thereof). Such compounds are known. See, e.g., U.S. Pat. Nos. 6,096,724; 6,844,434; and 5,260,291. Examples of alkylating agents useful for carrying out the present invention include [3H]-imidazo[5,1-d]-1,2,3,5-tetrazin-4-ones alkylating agents, particularly those of the general formula:

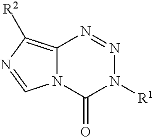

wherein R¹ represents a hydrogen atom, or a straight- or branched-chain alkyl, alkenyl or alkynyl group containing up to 6 carbon atoms, each such group being unsubstituted or substituted by from one to three substituents selected from halogen (i.e. bromine, iodine or, preferably, cblorine or fluorine) atoms, straight- or branched-chain alkoxy, (e.g. methoxy), alkylthio, alkylsullihinyl and alkylsulphonyl groups containing up to 4 carbon atoms, and optionally substituted phenyl groups, or R¹ represents a cycloalkyl group, and R² represents a carbamoyl group which may carryon the nitrogen atom one or two groups selected from straight- and branched-chain alkyl and alkenyl groups, each containing up to 4 carbon atoms, and cycloalkyl groups, e.g., a methylcarbamoyl or dimethylcarbamoyl group. When the symbol R¹ represents an alkyl, alkenyl or alkynyl group substituted by two or three halogen atoms, the aforesaid halogen atoms may be the same or different. When the symbol R¹ represents an alkyl, alkenyl or alkynyl group substituted by one, two or three optionally substituted phenyl groups the optional substituents on the phenyl radical(s) may be selected from, for example, alkoxy and alkyl groups containing up to 4 carbon atoms (e.g. methoxy and/or methyl group(s)) and the nitro group; the symbol R¹ may represent, for example, a benzyl or p-methoxybenzyl group. Cycloalkyl groups within the definitions of symbols R¹ and R² contain 3 to 8, preferably 6, carbon atoms. The compounds may be provided as salts or prodrugs, particularly alkali metal salts when R¹ is H. See, e.g., U.S. Pat. No. 5,260,291.

Temozolomide, in oral dosage form as 5 mg, 20 mg, 100 mg, and 250 mg capsules, is commercially available as TEMODAR™ from Schering Corporation, Kenilworth, N.J. 07033 USA.

Chemotherapeutic agents, such as alkylating agents, may be prepared in pharmaceutically acceptable formulations in like manner as described above, in the same or different formulation that contains a tumor vaccine, e.g., EGFRvIII peptide.

In a preferred embodiment, the chemotherapeutic agent, such as an alkylating agent, is administered in a cycle of daily doses for 3, 4, 5, 6 or 7 consecutive days. A suitable daily dose may be from 50, 100 or 150 mg/m²/dose, up to 200, 250 or 300 mg/m²/dose. This cycle may be repeated, e.g., every two, three, four or five weeks, for up to a total of 6, 8 or 10 cycles. The first dose in the first cycle of alkylating agent may be administered at any suitable point in time. In some embodiments the first dose of agent is administered up to two or four weeks before administration of the therapeutic antibody; in some embodiments the first dose of agent is administered at least two, four or six weeks following the administration of the therapeutic antibody. Additional schedules of administration may be included where additional therapeutic treatments such as external beam radiotherapy are also applied to the subject. Typically the agents will be administered within 2 months of each other.

Antibodies which inhibit the function or proliferation of Treg cells can be used as are known in the art. These may polyclonal or monoclonal antibodies. They may be antibody derivatives such as fragments or single chain antibody constructs. Daclizumab is one such antibody that is commercially available.

Optionally, the subject may also receive external beam radiotherapy. For example, external beam radiotherapy may be utilized for brain tumors such as glioblastoma. External beam radiotherapy is known and can be carried out in accordance with known techniques. The beam can be generated by any suitable means, including medical linear accelerators and Cobalt 60 external beam units. The radiation source can be mounted in a gantry that rotates around the patient so that a target area within the patient is irradiated from different directions. Before irradiation the treatment is typically planned on a computer using algorithms that simulate the radiation beams and allow the medical personnel to design the beam therapy. Numerous variations of external beam therapy that can be used to carry out the present invention will be readily apparent to those skilled in the art. See, e.g., U.S. Pat. Nos. 6,882,702; 6,879,659; 6,865,253; 6,863,704; 6,826,254; 6,792,074; 6,714,620; and 5,528,650.

External beam therapy is preferably administered in a series of sessions in accordance with known techniques, with the sessions preferably beginning two to four weeks after administration of the therapeutic antibody. For example, the external beam radiotherapy may be administered 3, 4, 5, 6 or 7 days a week, over a period of four, five, six or seven weeks, at a daily dose of 0.5 or 1 Gy, up to 2 or 3 Gy, until the total desired dose (e.g., 30 or 40 Gy, up to 50 or 60 Gy) is administered.

The delivered dose may be to an area including a margin of normal tissue (e.g., ˜1, 2 or 3 cm margin in all directions) around the tumor, or where the tumor or a portion thereof has previously been surgically removed, around the site of the tumor.

Where external beam radiotherapy is employed, the patient may receive an additional schedule of chemotherapeutic agent administration, different from that described above, at a somewhat lower dose, during the course of the radiotherapy. For example, the patient may receive daily doses of chemotherapeutic agent, e.g., alkylating agent in an amount of from 25 or 50 mg/m²/dose up to 100 or 125 mg/m²/dose daily during the course of the external beam therapy.

Examples of tumor antigens which can be used as anti-tumor vaccines include but are not limited to Cyclin-dependent kinase 4; β-catenin; Caspase-8; MAGE-1; MAGE-3; Tyrosinase; Surface Ig idiotype; Her-2/neu Receptor; MUC-1; HPV E6 and E7; CD52 Idiotype CAMPATH-1, CD20; Cell surface glycoprotein CEA, mucin-1; Cell surface carbohydrate Lewis^{x}; CA-125; Epidermal growth factor receptor; p185HER2; IL-2R; FAP-α; Tenascin; and metalloproteinases. EGFRvIII is exemplary of tumor-specific antigens. Cells which express these antigens can also be used as vaccines. Preferably the cells are killed prior to administration. The cells can be fractionated so that a fraction enriched for the tumor antigen is used as a vaccine. These antigens are merely exemplary and are not intended to be a comprehensive of the many useful antigens known in the art or which may be used. Tumor antigens can be delivered as peptides, conjugated or not to other moieties. Tumor antigens can also be delivered in transfected cells. The transfected cells can be dendritic cells, for example. The cells may be transfected with RNA encoding the tumor antigens.

Multiple preclinical model systems have demonstrated that the depletion of immune cell subsets can abrogate the efficacy of several types of immunotherapeutic approaches (Heimberger et al., 2003) indicating that chemotherapy administered during the effector stages of immunotherapy may be deleterious to efficacy. However, this does not preclude utilizing these agents together when appropriately timed to minimize the aforementioned effects. Furthermore, although applicants do not wish to be bound by any particular theory regarding mechanism of action, the depletion of certain effector cells, such as Tregs, may be a highly desirable outcome of chemotherapy yielding greater immunotherapeutic efficacy or may promote a desirable cytokine profile for adequate tumor control.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

T_{Regs} are increased in patients with GBM and constitutively express the high affinity interleukin-2 receptor (IL-2Rα). Treatment with an antibody that blocks IL-2Rα signaling functionally inactivates and eliminates T_{Regs} without inducing autoimmune toxicity in murine models. We hypothesized that daclizumab, a commercially-available, IL-2Rα-specific antibody would function identically.

A randomized phase II clinical trial assessed the effects of daclizumab in the context of the cancer vaccine, CDX-110, which is comprised of an EGFRvIII-specific peptide sequence linked to KLH. EGFRvIII is a constitutively activated and immunogenic mutation not expressed in normal tissues, but widely expressed in GBMs and other neoplasms. In patients with newly-diagnosed, EGFRvIII+ GBM, after resection and radiation/TMZ, patients received CDX-110 vaccinations biweekly×3, then monthly until tumor progression in combination with TMZ (200 mg/m²×5/28 days). Half the patients were randomized to receive daclizumab (1 mg/Kg×1) at the first vaccine. The others received saline in a double-blinded fashion.

There were no drug related serious adverse events. EGFRvIII-specific immune responses were generated in all patients, and all immune responses were sustained or enhanced during subsequent TMZ cycles. Preliminary analysis (n=4) suggests that daclizumab reduces T_{reg} (CD 4⁺CD25⁺CD45RO⁺FOXP3⁺) numbers [change 82.4±7.1% from baseline (p=0.011; t-test)] without reducing overall CD8⁺ or CD4⁺ T-cell counts. T_{Regs} decreased only 3.7+11.0% after vaccination in the saline treated group during the same interval. Preliminary analysis (n=4) also suggest that daclizumab enhanced EGFRvIII-specific immune responses (p=0.01; t-test) and enhanced the titer of cytotoxic EGFRvIII-specific IgG1 isotype antibodies compared to the saline treated group (p=0.003; t-test) and compared to previously vaccinated patients who did not receive daclizumab (p=0.0015; t-test). TTP (time to progression) and OS (overall survival) survival in both arms has not been reached.

Daclizumab may reduce T_{reg} counts in patients with GBM. TMZ and daclizumab may enhance EGFRvIII-targeted immune responses despite lymphodepletion.

### EXAMPLE 2

EGFRvIII-specific peptide vaccines induce cellular and humoral antitumor immunity and prolong survival in murine brain tumor model systems without inducing autoimmunity. Human DCs loaded with PEPvIII-KLH induce potent EGFRvIII-specific lysis and stimulate CD4+ and CD8+ T-cells to secrete high levels of γ-IFN.

Our Phase I and multi-institutional Phase II studies demonstrate that vaccinations with an EGFRvIII-specific peptide induce T- and B-cell immunity, produce nearly complete radiographic responses in all patients with residual tumor, and universally eliminate detection of EGFRvIII-expressing cells. Recurrent tumors, however, continue to express wild-type EGFR suggesting that the tumor-targeted immune response is specific, but productive intra-molecular cross-priming against other potential tumor-specific antigens is incomplete and relevant endogenous antitumor immune responses remain attenuated. Similar results were also obtained in pre-clinical murine studies. We believe that productive extension of such secondary immune responses is hindered by the presence of T_{Regs}. Unarmed IL-2Rα-specific antibodies given in vivo to mice during recovery from TMZ-induced lymphopenia functionally inactivate T_{Reg}, while dramatically enhancing vaccine-induced immune responses. Daclizumab, a humanized IL-2Rα-specific antibody, leads to a reduction of human T_{Regs} and to an increase in effector T-cell function in vitro.

Vaccination—Our human in vitro studies have shown that EGFRvIII is a suitable immunologic target for inducing T-cell proliferation, cytokine secretion, and specific lysis of GBM cell lines. Our preclinical studies, using a syngeneic murine homologue of the human EGFRvIII mutation, demonstrate that vaccination with an EGFRvIII-specific peptide (PEPvIII-KLH) induces cellular and humoral antitumor immunity and prolongs survival in murine brain tumor model systems without inducing autoimmunity, but with tumor recurrence due to antigen escape. We have demonstrated in murine models that conjugation to KLH and the use of GM-CSF as an adjuvant are critical to the immune response generated.

We have now completed two consecutive and one multi-institutional Phase II study with PEPvIII-KLH. Patients newly-diagnosed with GBM are vaccinated monthly after receiving standard radiation and TMZ therapy and are followed for clinical and radiographic responses every month. These studies have produced remarkably consistent and impressive survival results along with the induction of T- and B-cell immunity, induction of PEPvIII DTH responses (P=0.01, paired t-test), nearly complete radiographic responses in all patients with residual tumor, and have universally eliminated EGFRvIII-expressing cells in recurrent tumors. Recurrent tumors, however, continue to express wild-type EGFR suggesting that the tumor-targeted immune response is specific, but productive intramolecular cross-priming against other tumor-specific antigens is incomplete and relevant endogenous antitumor immune responses remain attenuated. We believe that productive extension of these immune responses is hindered by the presence T_{Regs}.

Patients with GBM exhibit increased TReg fraction and reversible Immunosuppression upon TReg Removal—We have found that patients with newly-diagnosed GBM exhibit an increased fraction of CD4+CD25+FoxP3+ T_{Regs} amongst a overall diminished CD4+ T-cell compartment compared to healthy individuals. Removal of T_{Regs} in vitro is able to reverse the well-characterized cell mediated immune defects described in these patients such that T-cells from patients with GBM exhibit equivalent proliferation and Th1 cytokine secretion profile as normal hosts in the absence of T_{Regs}. These findings suggest that the increased fraction of T_{Regs} present in patients with GBM constitute a significant, and importantly, reversible constituent of the immunosuppressed profile in these patients and that the removal or inhibition of TReg function may be a potent strategy to enhance the efficacy of immunotherapy.

Our murine astrocytoma model displays a parallel increase in T_{Regs} and immunosuppression can be reversed after TReg inhibition with an unarmed anti-IL-2Rα MAb61, validating the treatment strategy proposed here in a relevant preclinical model and demonstrating the importance of endogenous antitumor immune responses that can be unleashed by TReg abrogation.

TMZ-induced Lymphopenia and IL-2Rα Blockade Enhance Immune Responses—TMZ produces a survival benefit in patients with GBM172, and has become a routine part of the therapy for these patients. It also induces transient, Grade 3 lymphopenia (<500 cells/uL) in 70% of patients with GBM after the first cycle (150-200 mg/m2/5 d) with nadirs occurring 14-21 days after treatment (n=10). TMZ treatment also leads to an increase in the proportion of T_{Regs} in the peripheral blood (unpublished). Despite this, we have been able to induce and maintain potent EGFRvIII-specific humoral and cellular immune responses in patients with GBM receiving TMZ. Thus, we evaluated TMZ for its capacity to induce homeostatic proliferation and to enhance antigen-specific immune responses in a murine model. Markedly elevated levels of antigen-specific T-cells could be achieved and maintained in mice receiving antigen-specific vaccination after TMZ-induced lymphopenia when compared to normal hosts. Untreated mice exhibited an increase of OVAspecific T-cells to a mean of 1.90% of circulating CD8+ T-cells one week post vaccination which subsequently contracted to 0.40% after two weeks, while TMZ pre-treatment resulted in an expansion from a frequency of 0.40% to 10.64% (absolute count increase from 3.41 cells/uL blood to 42.61 cells/uL) that continued to expand for 4 weeks post-vaccination to 21.18%.

OVA-specific T-cells in TMZ treated and vaccinated hosts were shown to be Th1-type memory/effector cells by surface phenotype and cytokine secretion (CD44hiCD43loIFN-γ+). Anti-IL-2Rα treatment, while having a negative impact on T-cells responses in normal mice, as might have been expected, led to an even more dramatic enhancement of responses in TMZ treated mice (41.96%) indicating that during recovery from TMZ-induced lymphopenia, IL-2Rα blockade is differentially efficacious in enhancing effector T-cell responses. Analysis of lymph nodes, bone marrow, and spleen showed similar increases in TMZ treated mice in all compartments.

### EXAMPLE 3

The effect of daclizumab on the functional suppressive capacity of CD4CD25+CD127− T_{Regs} in patients with GBM during recovery from therapeutic TMZ-induced lymphodepletion.

Patient Population—Patients eligible for this study will meet the same criteria as our prior studies using PEPvIII-KLH (IRB #3108-03-9R2), and will be conducted under FDA-approved IND-9944. Adults with newly-diagnosed GBM who have obtained a definitive resection (defined as a 95% volumetric resection by Vitrea®) of a GBM expressing EGFRvIII (>1+ on >10%), a Karnofsky performance status >80, and a Curran40 score of I-IV will be eligible. Patients with evidence of leptomeningeal or multicentric disease, prior therapy other than external beam radiation therapy (RT) or TMZ, solid organ transplant, prior treatment with daclizumab, pregnant or breast feeding, active infection or an unexplained febrile illness, known immunosuppressive disease or HIV infection, or unstable or severe medical conditions will be excluded. 06-methylguaninie-DNA-methyltransferase (MGMT) status of the tumor will be assessed by PCR and IHC using methodology established at our institution for exploratory retrospective stratification of patient responses to TMZ.

Treatment Plan—Patients will be consented prior to eligibility screening, and all patient related procedures will be Health Information Privacy and Accountability Act compliant and approved by the IRB. Within 4 weeks of resection, patients will undergo standard external beam RT with concurrent TMZ at a target dose of 75 mg/m2/day. Patients with radiographic evidence of progressive disease, unable to tolerate TMZ, or dependent on corticosteroids above physiologic levels (2 mg dexamethasone) at time of first vaccination will be replaced. Remaining patients will then receive an initial cycle of TMZ at a standard targeted dose of 200 mg/m2/d for 5 days 3+1 weeks after completing RT and will be independently randomized to receive daclizumab (1 mg/Kg) or saline in a blinded fashion simultaneous with PEPvIII-KLH vaccine (SOP-JHS-HDC-CL-012 “Administration of Vaccination Procedure”) and ALT.

Prior to first vaccination, each patient will undergo a 2 hour leukapheresis (SOP-JHS-HDC-CL-023 “Leukapheresis Collection Procedure”) at the Duke Apheresis Unit for collection of samples to be used in immunologic monitoring and for harvesting of PBMCs for ALT. ALT will be used, as in our murine studies, to function as a population of responder cells prior to endogenous recovery in lymphodepleted hosts. Only the standard dose of daclizumab (1 mg/kg) currently approved by the FDA will be assessed. The decision to dose escalate in subsequent trials will be dependent on analysis of the safety and immunologic responses obtained in this trial.

Vaccine #2 and #3 will occur at subsequent 2 week intervals. Patients will then be vaccinated monthly in conjunction with subsequent TMZ cycles every 28 days for a total of 6 cycles after RT. TMZ will be given on days 1-5 with PEPvIII-KLH given on days 20+2 as lymphocyte counts begin to recover as described by us previously. Vaccinations will continue after TMZ cycles are finished. Patients will be imaged bimonthly without receiving any other prescribed antitumor therapy and continue with vaccinations until progression. Patients will be followed until death. Clinical monitoring for efficacy and toxicity is described below.

As part of standard care for these patients, upon tumor progression, participants may undergo stereotactic biopsy or resection. Tumor progression will need to be documented histologically, unless there are clinical contraindications, to exclude inflammatory responses presenting as radiographic or clinical changes, which could indicate a potentially toxic or therapeutic responses and not tumor progression. If tissue is obtained, it will be used to confirm tumor progression histologically and to assess immunologic cell infiltration and EGFRvIII antigen escape by IHC and rt-PCR at the tumor site. Patients will be eligible for additional adjuvant therapy at the time of tumor progression. These therapies and any subsequent serious adverse events will be documented.

Immunologic Monitoring—Prior to the 1st vaccine, after the 3rd vaccine, and monthly thereafter, all patients will be assessed for TReg suppressive activity and number, anti-EGFRvIII IgG and IgM titers, and for CD4+ and CD8+ T-cell responses. NK cells will also be collected for phenotypic and functional assays. From the leukaphereses, approximately 3×10⁹ PBMCs will be cryopreserved for more detailed immunologic monitoring and DC generation (SOP-JHS-HDC-CP-003 “Dendritic Cell Generation”) in our FDA-approved Cell Processing Facility, but simultaneous peripheral blood samples will also be obtained for comparative longitudinal studies. For the purposes of quality assurance, all samples will be batched and individual assays monitored for intra-assay variability by running existing control samples with each assay.

Statistical Analysis—We have devoted considerable effort to validating the immunologic assays outlined below for monitoring TReg function, antigen-specific responses in CD8+ or CD4+ T-cells, and NK cell functional activity in cancer patients. The reproducibility of these assays in our hands ensures that they can be reliably used for monitoring of changes after vaccination with and without daclizumab. Power calculations for this study are based on our primary endpoint which is functional suppressive capacity of CD4+CD25+CD 127− T_{Regs}. For these calculations, we assumed that measurements made in each treatment group at a given phytohemagglutinin (PHA) level would be normally distributed with standard deviation of a. Based on our experience with these assays within this patient population57, we estimate that with a sample size of 20 randomized patients we will have 80% power to detect a difference of 1.41σ at the 0.017 significance level (one-tailed). A 0.017 level of significance is used to adjust (Bonferroni correction) for the use of 3 non-zero PHA doses (0.1, 1, 10 μg/well) in the proliferation assay. Thus, with only 20 patients, we will have sufficient power to detect treatment effects much more subtle than the ones observed in our pre-clinical studies and effects that we believe would be clinically significant. However, because the magnitude and variability of the effects of daclizumab on T_{Regs} remain unknown in humans, once human data become available from these studies, power analyses will be recalculated and patient numbers adjusted if needed. If daclizumab treatment does not significantly inhibit the functional recovery of T_{Regs} (primary endpoint), we would not be interested in the further development of this drug as a vaccine adjuvant. Therefore, a one-sided hypothesis test is utilized. Power calculations are not provided for subsequent analyses as the number of patients in this trial will be determined by the primary endpoint, and the remainder of the analyses are exploratory and hypothesis generating only. With 10 patients in each arm, however, we will have 80% power to detect difference >1.325σ between groups for each assay at α=0.05.

TReg Functional Assays—In accordance with our first specific aim and our primary endpoint, the functional capacity of CD4⁺CD25⁺CD127⁻ T_{Reg} will be assessed, as described by us previously, and comparisons will be made between groups that randomly received saline or daclizumab. Briefly, leukapheresis samples are diluted, underlayered with Ficoll (Histopaque 1077, Sigma), and spun. Interphases are collected, washed, and subjected to a 2 h adherence step. A CD4+ T-Cell Isolation Kit II (Miltenyi) is used to isolate untouched CD4+ cells. αCD127-biotin (BD, #558633) is also added to the isolation cocktail to deplete CD127+ cells. CD25+ cells are further isolated using Miltenyi CD25− beads (#130-090-445). This approach yields a population >98% pure that has been shown by us to function as T_{Regs} in suppression assays. To verify suppressive capacity among isolated T_{Regs}, 5×104 CD4+CD25− responders are plated alone or at a 1:1 ratio with autologous T_{Regs} in complete T-cell media. After 72 h, levels of proliferation are assessed by ³[H]-thymidine incorporation over 16h then harvested on a FilterMate cell harvester and 3[H] counted. Means of triplicate wells are taken. As a subordinate assay, to verify anergy in the T_{Reg} population, 1×10⁵ T_{Regs} or CD4+CD25− cells are plated alone in triplicate wells using PHA as a stimulator and analyzed similarly.

Statistical Analysis—Our primary analysis will consist of a comparison of proliferative T-cell response to PHA among treatment groups (+/−daclizumab) using a generalized linear model for normal data that accounts for correlation of measurement replication across PHA doses (0, 0.1, 1, 10 μg/well) within subjects. This analysis determines whether the difference in treatment groups relative to proliferative response remains constant over PHA doses (i.e., curves remain parallel or not). A test of interaction with 3 degrees of freedom will be used. If the curves deviate significantly from parallelism, we will conclude that the effect of treatment varies as a function of PHA dose level. Within the context of this model, statistical contrasts will be used to assess treatment differences at specific PHA dose levels. If the test for interaction is not statistically significant, one will conclude that the treatment effect is constant over PHA dose levels. A generalized linear model without an interaction will be used to assess whether the main effect for treatment is statistically significant (i.e., treatment differences are non-zero). In this analysis and others, if assumptions required for t-tests are not satisfied, a nonparametric Wilcoxon rank sum test will be conducted at each PHA level. Significance for this and other assays will be taken at the α=0.05 level unless otherwise indicated. A Bonferroni correction will be used to control for the multiple comparisons.

Expected Results, Limitations, and Pitfalls—Given that our murine studies showed significant inhibition of TReg function when exposed to an IL-2Rα blocking MAb, and our preliminary in vitro studies with human _{TRegs} show enhancement of CD4+ T-cell responses in the presence of daclizumab, we expect to see reduced suppressive function in T_{Regs} isolated from patients treated with daclizumab as has recently been shown for a small group of patients with MS treated with daclizumab. These assays will be complemented by the quantitative studies described below and will be adjusted for cell number if TReg numbers are reduced. However, Gavin et al. demonstrated that when T_{Regs} are transferred into lymphopenic RAG1− mice, CD25 expression is lost. If it is found in our patients that during homeostatic reconstitution, after TMZ-induced lymphopenia, that recovering T_{Regs} lost or failed to express CD25, then one would not expect treatment with daclizumab to have any effect. However, Antony et al have subsequently shown that only when T_{Regs} are transferred in isolation does the loss of CD25 occur, presumably due to the lack of IL-2 producing helper T-cells. When T_{Regs} were transferred with helper cells, as would be the case with global homeostatic recovery after lymphopenia, then CD25 expression was maintained by the T_{Regs}. The presence of CD25 on T_{Regs} during homeostatic proliferation was also confirmed by Zelenay et al. Finally, we have preliminary data that clearly demonstrates that FOXP3+ T-cells express CD25 during recovery from TMZ-induced lymphopenia. It is also possible that an IL-2-independent pathway for peripheral TReg homeostasis is operative or that IL-2 signaling may be maintained sufficiently to promote expansion of T_{Reg}, not unlike the situation reported for neonatal T_{Regs} despite daclizumab treatment. If T_{Regs} are maintained and functional, increasing doses of daclizumab and IL-2 independent pathways will be investigated.

TReg Counts—As a secondary analysis, T_{Regs} levels will be determined before and after vaccination in each treatment group (+/−daclizumab), as described by us previously, in whole blood and paired leukapheresis samples using combinations of titrated antibodies against CD3 (UCHT1), CD4 (RPA-T4), CD8 (RPA-T8), CD45RO (UCHL1), CD127, and CD25 (BD Biosciences, San Jose, Calif.). Following incubation, Optilyse B (Immunotech, Marseille, France) is added. Cells are then re-incubated at RT for 15m and diluted with distilled water. For intracellular FOXP3 staining, cells are washed and incubated for 1h with Fix/Perm Buffer (eBioscience, San Diego, Calif.) and then washed and labeled with αFOXP3 (PCH101, eBioscience) for 30 minutes in the dark at 4° C. in the presence of Permeabilization Buffer (eBioscience). Samples were washed and analyzed on an ARIA flow cytometer (BD Biosciences). Data analysis is performed using BD FloJo software. To further explore the potential mechanism of daclizumab in this setting, internalization of CD25 on T_{Regs} after daclizumab will be examined using a non-overlapping antibody (MA251) that does not block the IL-2 binding site. Anti-human secondary F(ab′)₂ goat antibodies specific for both human IgG and IgM (Jackson ImmunoResearch, #109-116-127) that will recognize daclizumab bound to CD25 on the cell surface will also be used. This will provide data similar to that derived for our animal studies.

Statistical Analysis—Assuming normal data distribution, an unpaired t-test will be used to compare treatment groups (+/−daclizumab) relative to mean TReg levels after vaccination. Because patients will be randomly assigned to each group, and both will receive equal vaccinations, differences between groups after vaccination should be attributable to daclizumab. An unpaired t-test will be used to examine baseline differences in TReg levels, however. As an exploratory analysis, a paired t-test will also be used within groups to determine differences in TReg counts before and after vaccination.

Expected Results, Limitations, and Pitfalls—murine and monkey in vivo studies, along with our in vitro studies on human cells, all suggest that IL-2Rα blockade will result in a reduction in TReg numbers. However, Setoguchi et al. have shown that although IL-2 neutralization does reduce the number of FOXP3+ cells in the thymus and periphery of normal mice resulting in autoimmunity, it does not inhibit the lymphopenia-induced homeostatic expansion of T_{Regs} in a T-cell deficient environment. If this is found, the functional activities of these recovering T_{Regs} will be important to investigate and hence forms our primary endpoint. Our preliminary data from mice however, suggests that even in the context of increased TReg proportion after TMZ-induce lymphodepletion, homeostatic proliferative mechanisms favor the preferential expansion of T-cells stimulated by vaccination over that induced in non-depleted hosts and anti-IL-2Rα blockade significantly augments this effector Tcell expansion. Our central hypothesis is that daclizumab will reduce the suppressive function of extracerebral T_{Regs}. We believe that by inactivating peripheral T_{Regs}, vaccine induced and endogenous antitumor immune responses will be enhanced. Still, it remains possible that peripheral blood TReg counts will not be reflective of counts at the tumor site which may be equally or more important for ultimate clinical response.

Because we have had success in vaccinating patients in the minimally-residual disease setting, we have chosen this patient population for our studies. However, these patients do not have sufficient tumor to quantitate T_{Regs} during daclizumab therapy. Our pre-clinical results though would argue that anti-IL-2Rα antibody delivered systemically, which reduces peripheral blood T_{Regs}, is relevant to the intracerebral tumor response. Furthermore, patients with MS treated with daclizumab have a reduction in T_{Regs} in the cerebrospinal fluid that correlates with peripheral blood counts. Our human data also suggests a correlation between intratumoral and peripheral blood TReg counts. Finally, to address this question directly, we are planning a follow-up study that will treat patients with daclizumab at tumor recurrence and evaluate the differential effects on peripheral blood and intratumoral T_{Regs}.

Cytokine Analysis—As an exploratory analysis, CD4+ T-cells derived from patients in each group will be cultured in 96-well plates with PHA, PEPvIII, KLH, EGFR ECD, total tumor lysate, and autologous normal brain lysate. After 72 h, supernatants are harvested and processed in duplicate with a custom BioRad Bio-Plex 7-plex (IL-2, IL-4, IL-6, IL-10, IL-12 (p70), IFN-γ, TNF-α) Cytokine Reagent Kit (BioRad) to differentiate responses into TH1 and TH2 types on a Luminex 100 machine (Luminex Corporation, Austin, Tex.). Unknown cytokine concentrations are calculated by BioPlex Manager software using standard curves derived from a recombinant cytokine standard. Commercial ELISAs will be used to measure IL-7, IL-15, and TGF-β1-2.

Statistical Analysis—An unpaired t-test will be used to compare cytokine differences between treatment groups as described above.

Expected Results, Limitations, and Pitfalls—CD4+ T-cells in patients with GBM have a TH2 bias. Our published in vitro human studies demonstrate that immunosuppressive TH2 cytokine profiles could be reversed by TReg depletion. Therefore, we expect that daclizumab treatment will productively alter CD4+ T-cell cytokine phenotype in these patients. The limitation of such assays is that they may fail to detect important cytokine inter-relationships. These will be explored prospectively if such relationships appear likely.

EGFRvIII-specific and Endogenous CD4+ and CD8+ Immune Responses—EGFRvIII-specific humoral and cellular immune responses will be monitored. Evidence for intra- and inter-molecular epitope spreading and evidence for the induction of autoimmunity will also be sought. For cellular immune responses a polyfunctional analysis of Tcell function will be performed as previously described. Briefly, cryopreserved PBMC samples are thawed and rested overnight at 370 C/5% CO2 in RPMI media containing 10% fetal calf serum. Cells are adjusted to 2×10⁶/well and incubated with 1 μg/mL of each of the co-stimulatory MAbs αCD28 and αCD49d with or without stimulation with PEPvIII, KLH, EGFR ECD, total tumor lysate, and autologous normal brain lysate (when available)(2 μg/ml) in the presence of Brefeldin A (5 μg/ml Sigma-Aldrich, St. Louis, Mo.) monensin (1 μg/ml; Golgistop, BD Biosciences, San Diego, Calif.), and □CD107a-Alexa 680 for 5-6 hr at 370 C and 5% CO2. Following stimulation, cells will be treated with EDTA for 15 minutes at ambient temperature (AT, 18-22° C.). The cells will be washed, and stained with MAbs specific for CD4 (Cy5.5-PE), CD8 Qdot 705, CD45RO PE-TR, CD27 PE-Cy5, CD57 Qdot 605, CD14/CD19 (Cascade Blue) and a vital-dye reagent (LIVE/DEAD Fixable Violet Dead Cell Stain Kit for Flow Cytometry; Invitrogen Corp., CA) for 20 minutes at RT. After two washes, 1× BD FACS Lysing solution (BD Biosciences, San Jose, Calif.) will be added and samples will be incubated for 10m at RT. After one wash, 1× BD FACS Permeabilizing Solution 2 (BD Biosciences, San Jose, Calif.) will be added and samples incubated for 10 minutes at AT. After one wash, cells will be stained with αCD3 (Cy7-APC), αIFN-γ(FITC), αTNF-α (Cy7-PE), αMIP1β (PE), and αIL2 (APC) for 30 minutes on ice, washed, and fixed in PBS containing 1% formaldehyde (Sigma-Aldrich, St. Louis, Mo.). In all experiments, a negative control (αCD28/49d), and a positive control (SEB, 10 μg/ml, Sigma-Aldrich) will be included. The samples are acquired on a custom LSRII polychromatic flow cytometer (BD Immunocytometry System, San Jose, Calif.) equipped for detection of 17 fluorescent parameters. We are planning to collect a minimum of 500,000 total lymphocytes from each sample, because we expect the frequency of responding cells to be between 0.05 and 1.0%. This number of events is required based on calculations performed by Dr. Holden Maecker (BD Bioscience, personal communication) to detect a statistically significant number of positive events that can be used for the analysis of the data and the characterization of the different populations. Ideally, we would be able to select a validated surrogate immunologic response marker for clinical efficacy, but no such marker has been identified to date and studies to validate such a marker would need to be large and prospective and would be clearly beyond the scope of this proposal. It would be our intent, however, to incorporate any additional knowledge that becomes available at the time of data analysis to evaluate the relative biologic significance of the immune response markers that we have chosen. Tetramer studies are not performed because the HLA restriction of PEPvIII has not been determined except for HLA-A2 and we have not been successful in obtaining a functional version of this tetramer or a related pentamer. Tetramer anlaysis will be used, however, in patients with appropriate genetic haplotypes to quantitate changes in immunologic response to tumor-associated antigens differentially expressed in GBM before and after vaccination as follows: IL-13Rα₃₄₅₋₃₅₄, her-2₃₆₉₋₃₇₇, survivin₉₆₋₁₀₄, gp100₂₀₉₋₂₁₇, and TRP-2₁₈₀₋₁₈₈.

Statistical Analysis—The analysis used here will be one that has been successful at distinguishing HIV progressors and non-progressors based on T-cell phenotype. All data will be background subtracted. For each measure, a lower threshold corresponding to 2SD above background is set to 0 based on a Poisson model essentially allowing T-cells to be designated as positive or negative for a certain phenotypic marker. The number of positive phenotypic markers post-vaccination will be calculated for each patient. Treatment groups (+/−daclizumab) will be compared relative to the number of phenotypic markers observed postvaccination for each antigen using a Wilcoxon rank sum test. Additional exploratory analyses will be conducted using a Fisher's exact test that will compare treatment groups with respect to the proportion of patients with >4 positive markers. Correlations between phenotypic markers and EGFRvIII loss on recurrence will also be sought.

Expected Results, Limitations, and Pitfalls—We are not aware of any data that TReg depletion or IL-2Rα blockade alters the phenotype of CD4+ or CD8+ antigen-specific T-cell responses, but broad and multi-factorial T-cell responses have not been well-studied nor correlated with clinical results in cancer patients. Traditionally, IFN-γ responses alone by assays such as ELISPOT have been used to monitor cancer immunotherapy trials, but these results have not been rigorously correlated with clinical outcomes. Our approach relies on a broader set of immunologic parameters which have been correlated with clinical responses in other T-cells dependent diseases such as HIV. Although this study is not primarily powered to definitively establish immunologic correlations of clinical response, these exploratory investigations should provide novel data for hypothesis generation and both IFN-γ responses alone and more complex interactions will be available for exploratory study. It remains possible, however, that the analyses we have chosen will be specific for viral responses, and will not correlate with responses outcomes in cancer patients. Finally, the apparent immunologic editing of EGFRvIII expressing cells with this vaccination approach also offers a unique opportunity to explore the relationship between immune response and a clearly important clinical outcome.

Activation-induced Cell Death (AICD) and Proliferation (Ki67) Assays—The frequency of proliferating versus apoptotic cells will be determined by intracellular expression of the proliferation marker Ki67 or active Caspase 3 in T-cell subsets, for each treatment group, respectively. PBMCs are stained for surface expression (CD3/CD4/CD8/CD25), washed, fixed with FACS permeabilizing solution (Becton Dickinson), incubated with saturating amounts of anti-Ki67-FITC (Beckman-Coulter) and anti-active Caspase 3 ((BD Pharmingen) (30 mins @ 4° C.), and then washed and resuspended with 0.4% paraformaldehyde. Multi-color flow cytometric analysis will performed on a BD LSRII flow cytometer (Becton Dickinson).

Statistical Analysis—All data will be background subtracted and compared between treatment groups using upaired t-tests as described above.

Expected Results, Limitations, and Pitfalls—As described above, the main function of IL-2 appears to be the control of peripheral tolerance and regulation of the peripheral lymphoid compartment by influencing the balance between clonal expansion and AICD. IL-2 has also been shown to be important during the T-cell death phase resulting in increased survival and proliferation of antigenspecific T-cells23. Williams et al., however, recently used mixed bone marrow chimeras (WT/IL-2Rα^{-/31} ) to investigate the role of IL-2 signaling in mice with a full complement of T_{Regs}. Although they found that the frequency of cells expressing CD62L and IL-7Rα(consistent with a central memory phenotype which has been shown to correlate with high proliferative and protective capacity) higher in the IL-2Rα^{-/-} cohort, they demonstrated that an IL-2 signal during the primary immune response was required for survival and accumulation of dividing cells during secondary antigen encounter. This represents a previously unappreciated role for IL-2 during the primary immune response that may be significantly negatively influenced by treatment with an IL-2Rα blocking MAb such as daclizumab. However, the balance between TReg inhibition and this phenomenon will be better appreciated by our studies as will the potential ability of other cytokines signaling through the shared βγ receptor, such as IL-7 or IL-15, present in abundance during lymphopenic recovery, to overcome this IL-2 deficit.

EGFRvIII-specific Humoral Responses—Subordinate analyses will explore similar relationships to those described above for humoral responses. Briefly, antibody levels to specific antigens will be determined by FACS analysis of antigen immobilized on beads. Beads are reacted with serum, washed, and developed with fluorochrome conjugated polyclonal anti-human secondary. Bead performance is validated by comparing serum reactivity of antigen-specific beads to serum reactivity with antigen transfected cells. As another means of verification, sample serums will be pre-incubated with excess specific and non-specific antigens, insuring that human antibody binding is specific.

Statistical Analysis—All data will be background subtracted and compared using upaired t-tests as described above.

Expected Results, Limitations, and Pitfalls—This assay has been thoroughly validated in our laboratory, and robust αEGFRvIII antibody responses have been generated previously using this vaccine approach. This proposal should provide further data on the potential role of antibodies on intra- and inter-molecular spread of the induced immune response.

Delayed Type Hypersensitivity—DTH tests will be performed at baseline and 2 weeks after the 3rd immunization and every two months thereafter. For skin testing, PEPvIII, KLH and standard recall antigens are injected intradermally (SOP-JHS-HDC-CL-014, “Delayed Type Hypersensitivity Testing and Biopsy Procedure”). Induration will be recorded 48-72 hours later and >10 mm of induration will be considered a positive response.

Statistical Analysis—Assuming that all patients will have no response before vaccination, each patient will be classified as a responder or nonresponder based upon an induration at the response site >10 mm. Fisher's exact test will be used to compare responses between groups after each vaccination.

NK Cell Phenotype, Function, and Cytotoxicity—NK cells play a pivotal role in early immune responses and have been shown to be activated by DC immunization. They have also been shown to kill activated T-cells. To assess these functions NK cells are sorted (CD3−CD56+) using a FACSAria. CD56^{bright} cells are further differentiated by being CX3CR1− (MBL International). For cytokine analysis, purified populations (>95%) are left unstimulated or stimulated with IL-12 (20 ng/mL) and IL-15 (100 ng/mL) (PeproTech) for 72 h. Supernatants are analyzed as described above. To evaluate for the effects of NK cells on CD4+ and CD8+ proliferation overall and in an antigen specific manner, PBMCs at baseline and after daclizumab therapy in vivo will be obtained from peripheral blood, labeled with CFSE (Molecular Probes, 1 μM), and activated either polyclonally with plate bound CD3/CD28 or with the specific antigens PEPvIII, KLH, EGFR-ECD, or normal brain or tumor lysate for 72 h in the presence of absence of daclizumab (matched for sample) as previously described. NK cells will be removed with CD56 beads (Miltenyi Biotec) for paired samples. The samples are then washed and re-seeded in media with IL-7 and IL-15. The numbers of T-cells and NK cells is analyzed. The effect of daclizumab therapy on the cytotoxicity of NK cells against resting or activated T-cells and K562 cells will be tested using standard ⁵¹Cr release assays as described.

Statistical Analysis—All analyses will be conducted as described above for T-cells.

Expected Results, Limitations, and Pitfalls—Daclizumab has been successfully used to inhibit disease activity in patients with MS who fail to respond to interferon γ and in patients with active uveitis. These effects took place gradually over several months. Bielekova et al. found, that in patients with MS, treatment with daclizumab in vitro lead to diminished T-cell survival and an expansion of NK cells. Removal of NK cells from the cultures completely restored T-cell survival and function suggesting that daclizumab had no direct detrimental effect on effector CD4+ or CD8+ T-cells. The NK cell effect was dependent on cell-cell contact and the expansion of CD56^{bright} NK cells and correlated with a loss of CD4+ and CD8+ T-cells in vitro. Daclizumab therapy enhanced the cytotoxicity of resting (non-IL-2 activated) CD56^{bright} NK cells against activated T-cells. However, other than the increase in numbers of NK cells, there was no difference in CD4+ or CD8+ T-cell proliferation or IL-2 production in the groups treated with daclizumab in vivo in the absence of additional daclizumab added in vitro. Interestingly, daclizumab did enhanced CD8+ T-cell and NK cell responses to IL-15. We believe that in patients with ongoing inflammatory responses such as those with organ transplantation, MS, or uveitis that maintenance of the inflammation may be dependent on signaling through the high affinity IL-2Rα. The upregulation of IL-2Rα with established immune responses has been well demonstrated.

Our preliminary data indicates that the situation may be quite different in predominantly immunosuppressed tumor patients that are undergoing lymphocyte reconstitution. The continuous dosing schedule used to treat MS and uveitis is also much different than we propose.

Detection of EGFRvIII by Immunohistochemistry and rt-PCR—For IHC, sections (6 μm) are deparaffinized with xylene, blocked for endogenous peroxidase (3% H2O2) and Fc receptor (Innovex Biosciences) before the addition of MAb. Horseradish peroxidase detection (BioGenex) is used with MAb specific for EGFRvIII (L8A4)195 and isotype control (murine IgG1; R&D Systems). Post-fixation retrieval, and incubation time are established for each MAb using DAB (Innovex Biosciences). EGFRvIII gene expression level is analyzed using Quantitative RT-PCR67. Total tumor RNA is isolated from patient's paraffin embedding brain tissue with a kit “RecoverAll™ Total Nucleic Acid Isolation” (Ambion) according to manufacture's instruction. 1st strand cDNA is synthesized from each total RNA with SuperScriptase III (Invitrogen) by following manufacture's protocol & diluted 25-folds before Real-time PCR (qPCR). Then EGFRvIII gene expression level is measured with qPCR on ABI 7900HT Real Time PCR System (Applied Biosystems) at annealing temperature 60° C. by gene specific primers and probe (5′-CTG CCC GGC GAG TCG3′ (SEQ ID NO: 1), 5′-CCG TCT TCC TCC ATC TCA TAG C3′ (SEQ ID NO: 2), and 5′FAM-AAG GTA ATT ATG TGG TGA CAG ATC ACG GC-TAMRA3′; SEQ ID NO: 3) in TaqMan Universal Master Mix (Applied Biosystems) and normalized with Human GAPDH Endogenous control kit (VIC-MGB) in TaqMan Mix (Applied Biosystems). All recovered samples will be sequenced to uncover any novel escape mutations in EGFR or EGFRvIII as previously described (See Letter of Collaboration).

Clinical Monitoring—Patients will be assessed before each vaccination by general, neurologic, and ophthalmologic examination; brain MR imaging; and a panel of clinical laboratory analyses to screen for the development of subclinical autoimmunity. The most common manifestations of autoimmunity seen in related trials have included enterocolitis, dermatitis, uveitis, hepatitis, and hypophysitis. Serum thyroglobulin Ab, rheumatoid factor, antinuclear Ab82; human anti-human (anti idiotypic) Ab (to daclizumab), erythrocyte sedimentation rate, antinuclear Ab, thyroid-stimulating hormone, ACTH, cortisol, free T4, ALT, and AST will be obtained prior to each vaccine. If cerebrospinal fluid becomes available, it will be checked for oligclonal banding. Other clinically evident toxicity will be defined using the NCI Common Toxicity Criteria (3.0).

Safety and Statistical Analysis—Given the lack of significant toxicity in our prior multi-institutional EGFRvIII-targeted immunotherapy trials and those of others in patients with GBM204, and the established safety of the clinically-approved drug daclizumab, and the safety of other clinical approaches to TReg inhibition that have been used recently, we believe that it is likely that no significant toxicity will occur. However, any irreversible Grade 3 or reversible Grade 4 toxicity or life-threatening event, prior to tumor progression, will be considered a limiting toxicity. Patients will be randomized to each group in blocks of 3 and patients will be evaluated for toxicity within these groupings. If no patients in a given cohort experience a toxicity within 2 weeks another cohort will be enrolled within that arm. If 1 out of 3 patients in a given cohort experience DLT, 3 additional patients will still be entered in that arm, but if in any cohort of 3 or 6 consecutive patients, 2 patients develop a DLT, no further enrollment will be permitted and the study will be closed. Because we have previously safely treated many patients with the vaccine alone, toxicity in each arm will be evaluated independently. We have an existing Data and Safety Monitoring Plan approved by the NCI (See Appendix of Clinical Protocol) for use in these studies. For clinical and laboratory screening tests of autoimmunity, any change from a normal to an abnormal value or a 2 fold change will be considered a positive result. A patient with any “positive” test will be considered positive and the number of positive tests will not be weighted. A 2×2 table will then be constructed to differentiate the frequency of positive patients by treatment group. A Chi2 test will then be used to assess if these frequencies are statistically significant. Although not powered for survival analyses, the product limit estimator of Kaplan and Meier will be used to describe the distribution of survival time and TTP for both treatment groups, and the Cox proportional hazards model will be used to compare TTP and survival between the groups and with appropriate matched historical control groups. We would consider daclizumab worthy of further evaluation only if it did not significantly reduce TTP or overall survival relative to the control group and relative to the historical controls. Therefore, in the proposed study, we are interested in determining whether either treatment regimen has comparable or better results than our best contemporaneous results at Duke University which are with the use of radiolabeled MAbs delivered intratumorally. The most recent Phase II trial evaluating radiolabeled MAbs at Duke in patients with newly-diagnosed GBM had a median survival of 79.4 weeks, and the lower limit of the 95% confidence interval was 61.4 weeks. Assuming that survival is exponential in this context, a median of 61.4 weeks translates into a 6-month survival of approximately 75%. Therefore if 6-month survival is not definitively <75%, there would remain interest in further development of the regimen proposed here. With 10 patients in each arm of our trial, one can conduct a test with a=β=0.1 to differentiate between a 6-month survival rate of 35% and 75%. That means that if the true 6-month survival rate were <35% there is <10% chance of concluding that the treatment regimen is worthy of pursuing with further investigation. Similarly, if the true 6-month survival rate were >75% or more there is <10% chance of rejecting the treatment. Other comparison groups that could be used include a historical control database, the recently published Phase III trial of the regimen of TMZ to be used in this trial, or our previous trial dataset using this vaccine approach which had median survivals (95% CI) of 14.7 (12,0, 17.4), 14.6 (10.9, 21.2), and >29.8 months, respectively. In addition, we will assess clinical efficacy by estimating the proportion of patients who survived longer than expected according to Curran's recursive partition analysis.

### EXAMPLE 4

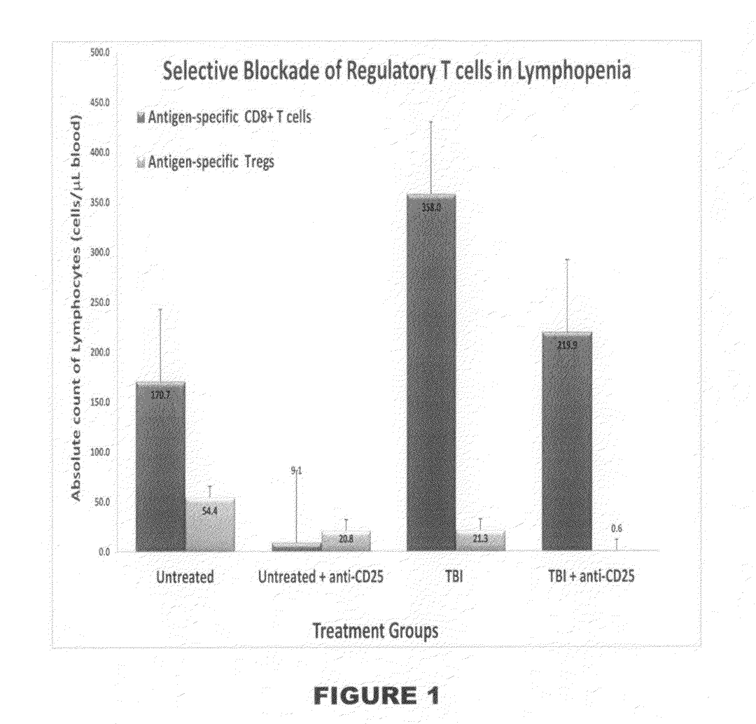

Selective blockade during lymphopenia of regulatory T cells using anti-CD25 monoclonal antibodies: C57BL/6 mice were either untreated or lymphodepleted using total body irradiation (TBI) prior to adoptive transfer of activated OVA-specific T cells with or without intravenous administration of anti-CD25 monoclonal antibody blockade (anti-CD25). Persistence of activated antigen-specific T cells (CD8+ tetramer+cells) and regulatory T cells (CD4+CD25+FOXP3+ cells) was evaluated by flow cytometry. Results demonstrate that only in context of lymphodepletion (TBI) are antigen-specific T cell responses maintained while in untreated mice antibody treatment impaired antigen specific immune responses. Regulatory T cells were effectively depleted in both normal and lymphopenic hosts. We have observed similar selective blockade of regulatory T cells using anti-CD25 monoclonal antibodies in combination with adoptive lymphocyte transfer or vaccination using chemotherapy-induced lymphodepletion. See FIG. 1.

We claim:

1. A method of treating a lymphodepleted subject, comprising:
administering to the lymphodepleted subject an inhibitory antibody to IL-2Rα sufficient to inhibit Treg cells, whereby an immune response to a specific antigen is increased.

2. The method of claim 1 wherein the immune response is a cellular response.

3. The method of claim 1 wherein the immune response is a humoral response.

4. The method of claim 1 wherein the subject has been subjected to total body irradiation to induce lymphodepletion of the subject.

5. The method of claim 1 wherein the subject has been lymphodepleted by administration of an antibody which specifically binds to lymphocytes.

6. The method of claim 1 wherein the subject has been immunized with the specific antigen as an immunogen.

7. The method of claim 1 wherein the subject has a disease which induces lymphodepletion of the subject.

8. The method of claim 7 wherein the disease is acquired immune deficiency syndrome.

9. The method of claim 7 wherein the disease is leukemia.

10. The method of claim 1 wherein the antibody is daclizumab.

11. The method of claim 1 wherein the antibody is basiliximab.

12. The method of claim 1 wherein the specific antigen is associated with glioblastoma multiforme.

13. The method of claim 1 wherein the subject has glioblastoma multiforme.

14. The method of claim 1 wherein lymphodepletion of the subject is not caused by a chemotherapeutic agent.

15. The method of claim 1 wherein the specific antigen is not a tumor antigen.

16. The method of claim 1 wherein the inhibitory antibody is administered at nadir or early recovery from nadir of lymphodepletion.

17. The method of claim 6 wherein the specific antigen and the inhibitory antibody are administered concomitantly.

18. The method of claim 6 wherein the specific antigen and the inhibitory antibody are administered sequentially.

19. A kit for use in subjects lymphodepleted by disease or irradiation comprising: an inhibitory antibody to IL-2Rα and an immunogen.

20. The kit of claim 17 which comprises no chemotherapeutic agent.

21. The kit of claim 17 which comprises no temozolomide.
